(19) United States
(12) Plant Patent Application Publication
(10) Pub. No.: US 2026/0363157 P1
(43) Pub. Date: Nov. 12, 2026

(54) Pelargonium Plant Named 'KLEPZ25946'

(50) ;

(71) Applicant: KLEMM + SOHN GMBH & CO. KG, Stuttgart (DE)

(72) Inventor: Nina Neu, Weissach im Tal (DE)

(73) Assignee: KLEMM + SOHN GMBH & CO. KG, Stuttgart (DE)

(21) Appl. No.: 19/655,175

(22) Filed: Apr. 22, 2026

(30) Foreign Application Priority Data
May 6, 2025 (CH) 25-3674

(51) Int. Cl. A01H 6/42; A01H 5/02

(52) U.S. Cl. PLT/327

(57) ABSTRACT

A new pelargonium plant particularly distinguished by deep orange flowers; and with green peduncles with no anthocyanin; is disclosed.

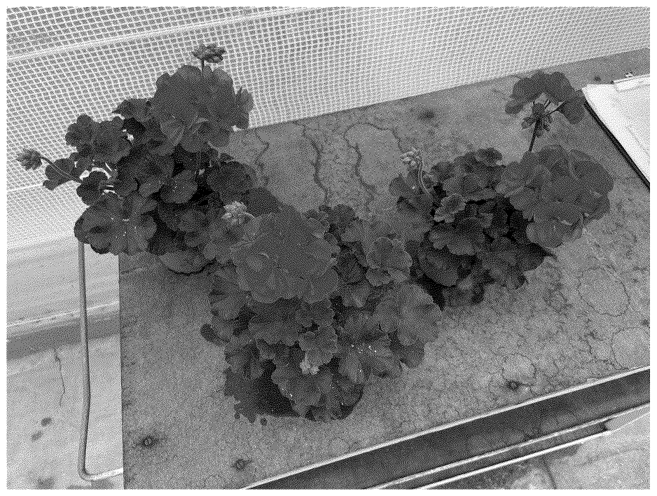

Genus and species: Pelargonium zonale.

Variety denomination: ‘KLEPZ25946’.

### CROSS REFERENCE TO RELATED APPLICATION

This patent application claims the benefit of priority from Plant Variety Protection Certificate No. 25-3674 as filed on May 6, 2025, in Switzerland, the contents of which is incorporated herein by reference for all the reference teaches and discloses.

### BACKGROUND OF THE NEW PLANT

The present invention comprises a new and distinct variety of pelargonium, botanically known as Pelargonium zonale and hereinafter referred to by the variety name ‘KLEPZ25946’. ‘KLEPZ25946’ originated from a controlled cross in July 2020 in Juja, Kenya between the female pelargonium variety ‘PZ-2019-9376’ (unpatented) and the male pelargonium variety ‘Ircoblan’ (unpatented).

Seeds from the controlled cross were obtained, sown and plants were grown for evaluation. A single plant was selected in June 2021 and subsequently named ‘KLEPZ25946’. In July 2021, ‘KLEPZ25946’ was first vegetatively propagated by vegetative stem cuttings in Stuttgart, Germany. ‘KLEPZ25946’ was found to reproduce true to type in successive generations of asexual propagation via stem cuttings in Stuttgart, Germany.

### SUMMARY

The following are the most outstanding and distinguishing characteristics of this new variety when grown under normal horticultural practices in Stuttgart, Germany.
1. Deep orange flowers; and
2. With green peduncles with no anthocyanin.

### DESCRIPTION OF THE PHOTOGRAPH

This new pelargonium plant is illustrated by the accompanying photograph which shows an overall view of the plant in a pot. The photograph is of an approximately 10-week-old plant from potting grown indoors in Stuttgart, Germany. The photograph was taken indoors in Stuttgart, Germany in April 2026. The colors shown are as true as can be reasonably obtained by conventional photographic procedures.

### DESCRIPTION OF THE NEW VARIETY

The following detailed descriptions set forth the distinctive characteristics of ‘KLEPZ25946’. Data was collected on 10-week-old plants from potting in Stuttgart, Germany grown under greenhouse conditions in April 2026. Color references are to The R.H.S. Colour Chart of The Royal Horticultural Society of London (R.H.S.), 5^{th} edition (2007).
Classification:
   Family.—Geraniaceae.
   Botanical.—Pelargonium zonale.
   Common.—Pelargonium.
   Designation.—‘KLEPZ25946’.
Parentage:
   Female parent.—‘PZ-2019-9376’ (unpatented).
   Male parent.—‘Ircoblan’ (unpatented).
Plant:
   Form.—Bushy.
   Habit.—Upright.
   Height (from top of soil).—11.0 cm to 14.0 cm.
   Width (horizontal plant diameter).—20.0 cm to 24.0 cm.
   Natural flowering season.—March to November.
   Propagation type.—Vegetative by stem cuttings.
   Time to produce a finished flowering plant.—9 weeks.
   Time to initiate and develop roots.—3 to 4 weeks.
   Root description.—Fine, dense, white, 164C.
   Flower type.—Zygomorphic.
Stems:
   Quantity of stems per plant.—3 to 4.
   Color.—144A.
   Surface texture.—Pubescent.
   Stem form.—Round.
   Diameter.—0.7 cm to 0.9 cm.
   Length.—6.0 cm to 10.0 cm.
   Internode length.—1.5 cm to 2.5 cm.
   Anthocyanin.—None observed.
Leaves:
   Arrangement.—Alternate.
   Shape.—Reniform.
   Apex.—Round.
   Base.—Open.
   Margin.—Crenate.
   Surface texture both surfaces.—Pubescent.
   Length.—6.5 cm to 7.5 cm.
   Width.—7.0 cm to 8.5 cm.
   Color.—Upper surface: N137A. Lower surface: 146A.
   Zonation.—Very weak, starts in 1.3 cm to 1.5 cm distance from leaf base, end not measurable as it fades out.
   Zonation color.—147A.
   Depth of Sinus.—1.9 cm to 2.3 cm.
   Petiole.—Length: 4.4 cm to 4.8 cm. Diameter: 0.3 cm to 0.4 cm. Color: 144A.
   Venation pattern.—Palmate.
   Venation color.—Upper surface: N137A. Lower surface: 144A.
Inflorescence:
   Lastingness of flowers on the plant.—6 to 8 days.
   Umbels.—Quantity of umbels per plant: 1 to 2 open with 2 closed buds. Length: 5.0 cm to 5.8 cm. Diameter: 11.5 cm to 12.5 cm.
   Individual florets of the umbel.—Quantity of florets per umbel: 14 to 20. Depth: 1.5 cm. Diameter: 5.0 cm.
   Fragrance.—Absent.
Flower buds:
   Cluster shape.—Round.
   Cluster size.—Length: 2.0 cm to 2.5 cm. Width: 2.5 cm to 2.7 cm.
   Surface texture.—Pubescent.
   Bud.—Height: 0.7 cm to 1.2 cm. Width: 0.5 cm to 0.8 cm. Shape: Elliptic. Apex: Acute. Base: Obtuse. Margin: Entire. Color: 144A.
Petals:
   Flower type.—Zygomorphic.
   Quantity per flower.—6 to 9.
   Shape.—Spatulate.
   Apex.—Round.
   Base.—Obtuse.
   Margin.—Entire.
   Length.—2.9 cm.
   Width.—2.5 cm.
   Surface texture.—Upper surface: Smooth. Lower surface: Smooth.
   Color of lobes.—Upper lobes: Upper surface: Base color N30A, small spot and veins at petal base 50A (diameter of spot 0.6 cm), petal base N155B (just the tip of the base is white) Lower surface: 40A. Lower lobes: Upper surface: Base color N30A, small spot at base of petal 42A (diameter of spot 0.5 cm), petal base N155B (just the tip of the base is white) Lower surface: 40A.
Petaloids:
   Quantity per flower.—3 to 4.
   Shape.—Spatulate with crippling.
   Apex.—Round.
   Base.—Obtuse.
   Margin.—Entire.
   Length.—1.7 cm to 2.3 cm.
   Width.—1.4 cm to 1.7 cm.
   Surface texture.—Upper surface: Smooth. Lower surface: Smooth.
   Color.—Upper surface: N30A. Lower surface: 40A.
Sepals:
   Number per flower.—5.
   Shape.—Lanceolate.
   Apex.—Acute.
   Base.—Obtuse.
   Margin.—Entire.
   Surface texture.—Upper surface: Smooth. Lower surface: Pubescent.
   Color.—Upper surface: 146A, 172A at base. Lower surface: 146A, 185A at base.
   Length.—1.3 cm.
   Width.—0.3 cm.
Pedicel:
   Length.—2.1 cm to 2.6 cm.
   Diameter.—0.2 cm.
   Color.—187A at connection to sepals, N199A at connection to stem.
   Surface texture.—Pubescent.
Peduncle:
   Length.—11.5 cm to 12.5 cm.
   Diameter.—0.4 cm.
   Color.—144A.
   Texture.—Pubescent.
Reproductive organs:
   Stamens.—Quantity per flower: 3 to 5. Anther length: 0.2 cm. Filament length: 0.7 cm. Filament color: N155A at base, 50B at tip. Pollen amount: Abundant. Pollen color: 169A.
   Pistils.—Number: 1. Stigma color: 46A. Style length: 0.3 cm. Style color: 50B. Ovary arrangement: Central. Ovary color: 147B. Ovary diameter: 0.2 cm. Ovary length: 0.4 cm. Ovary Texture: Strongly pubescent.
Fruit and seed set: None observed.
Disease and pest/insect resistance: None observed.

### COMPARISON WITH PARENTAL LINES AND KNOWN VARIETY

‘KLEPZ25946’ is distinguished from its parents as described in Table 1.

TABLE 1
Comparison with Parental Lines

| | | Female | Male |
|---|---|---|---|
| Characteristic | ‘KLEPZ25946’ | parent | parent |
| Flower color | Deep orange | Red | Light orange |
| Color of | Green with no | Dark | Light brown |
| Peduncles | anthocyanin | brown | |

‘KLEPZ25946’ can be compared to the commercial variety ‘KLEPZ15395’ (U.S. Plant Pat. No. 28,846). Differences between the two varieties are described in Table 2:

TABLE 2
Comparison with Similar Variety

| | Variety | Commercial |
|---|---|---|
| Characteristic | ‘KLEPZ25946’ | line |
| Flower Color | Deep orange | Light orange |
| Peduncle Color | Green | Brown |

I claim:

1. A new and distinct variety of pelargonium plant named ‘KLEPZ25946’ as illustrated and described herein.
